(19) United States
(12) Patent Application Publication
(10) Pub. No.: US 2026/0369404 A1
(43) Pub. Date: Nov. 19, 2026

(54) SYSTEMS AND METHODS FOR GUIDED DIAGNOSTIC TESTING AND CLOUD-BASED ANALYSIS

(71) Applicant: QUALISURE DIAGNOSTICS INC., Calgary (CA)

(72) Inventors: Oliver Bathe, Calgary (CA); Farshad Farshidfar, San Mateo, CA (US); Cynthia Stretch, Calgary (CA)

(73) Assignee: QUALISURE DIAGNOSTICS INC., Calgary, AB (CA)

(21) Appl. No.: 19/155,355

(22) PCT Filed: Feb. 10, 2023
(86) PCT No.: PCT/CA2023/050173

§ 371 Date: Aug. 11, 2025

(51) Int. Cl. G16H 50/20; G16B 20/00; G16H 10/20; G16H 10/40; G16H 15/00; G16H 30/40

(52) U.S. Cl. CPC G16H 50/20; G16B 20/00; G16H 10/20; G16H 10/40; G16H 15/00; G16H 30/40

(57) ABSTRACT

A diagnostic testing and analysis system for operating on a network, which may be a cloud-based architecture, includes a user terminal for obtaining one or more user instructions, providing recommendations for diagnostic tests based on user input, a central hub for accessing and ordering one or more tests, two or more data pre-processing modules (DPPMs), each DPPM for receiving raw medical test data and converting the data to a data matrix, an analysis engine for interpreting data from each of the data matrices, and producing a report comprising the diagnostic test result and the recommendations, where the user terminal, the clinical test recommendation tool, the DPPMs, and the analysis engine each comprise an interface to communicate over the network and the user can order one or more medical tests.

### FIELD OF THE DISCLOSURE

The present disclosure relates generally to medical diagnostic and prognostic systems, and in particular, to such systems having integrated recommendation tools and cloud-based analysis capabilities.

### BACKGROUND

Commonly, diagnosis and prognosis of disease are based on an analysis of a constellation of features such as symptoms, physical signs, laboratory abnormalities and radiographic data or images. When a specific diagnosis cannot be derived, the constellation of clinical manifestations is called a syndrome. Based upon the diagnosis and prognosis, treatments are administered with response to treatments being highly variable. One reason for variable treatment response is that a syndrome may actually represent a manifestation of a number of diverse disease processes with heterogeneous pathogenic mechanisms. For example, arthritis can be caused by multiple etiologies, and each etiology may have a number of subtypes. While there are therapies that may be effective in almost any form of arthritis (e.g., prednisone), the optimal treatment for each subtype would rely on knowledge of the underlying pathogenesis.

The scientific basis of disease pathogenesis and sub-classification has advanced owing to improved clinical research, development of multi-parametric analytical technologies, and increasingly sophisticated radiographic studies.

Precision diagnostics that arise from such converging technologies frequently rely on highly dimensional input data such as clinical and pathological data, molecular data from various tissues, and digital radiographic data. For example, there are a number of prognostic tools that require knowledge of a number of clinical and laboratory data; mutation patterns in cancers provide knowledge of potentially actionable therapeutic targets; diseases can be identified based on patterns of circulating metabolites or proteins; subtle physical and radiographic features of a mass can help to distinguish benign and malignant lesions. Such complex biomarkers enable a more precise sub-classification of disease processes. On the other hand, they are also more difficult to interpret, and few tools exist that enable rapid interpretation of clinical, molecular and radiographic data for important diagnostic signatures. Processing complex datasets associated with precision diagnostics often requires the use of machine learning (ML) methods, the implementation of which may vary widely in their structure, capability, and methods for disease sub-classifications. These implementations may require substantial processing power and computational resources, and this issue may be particularly evident in applications involving large numbers of diverse samples processed in parallel.

Diagnostic providers are faced with a number of challenges related to bringing their products to practical use. There is a rapid expansion of available diagnostic tests, many based on complex biomarkers. Physicians are unable to keep current with available tests. Academic and commercial entities are continually discovering and validating new biomarkers, and biomarkers with the greatest clinical and economic utility are commercialized. The accelerating rate of commercial biomarker development makes it difficult for physicians to remain current. Physicians need to know what tests are available that guide their clinical management. They must have access to information on each test's performance as well as access to evidence of a test's clinical utility to enable knowledgeable interpretation of test results and cost-effective test selection.

Similarly, patients have difficulty navigating the many diagnostic offerings. Frequently, patients do their own research to identify diagnostic tests that may facilitate their medical care. Given their often-limited medical knowledge, the process of finding and selecting tests that are valid, reliable and impactful on their clinical care can be daunting. Costs of health care are increasing, partly as a result of complex diagnostic tests. While common laboratory tests are relatively inexpensive, newer tests based on complex biomarkers are extremely expensive. Therefore, there is a need to ensure that any test paid for by the healthcare system or the patient is clinically impactful. Additionally, if a number of these more expensive tests are required, it would be cost efficient to bundle related tests (based on analytical platform and sample type, for example).

Diagnostic testing is becoming more fragmented, most precision medicine tests are performed in centralized labs. Along with the rapid increase in the number of biomarkers comes the rapid proliferation of companies focused on biomarker development. While the introduction of multiple parties accelerates progress, it also introduces fragmentation. Therefore, for example, it is conceivable that, in the current environment, for the complete molecular characterization of a tumor, one may need to engage multiple commercial entities to determine the molecular subtype of a tumor, its propensity to recur after surgery, its likelihood of spreading to different areas of the body, and its sensitivity to various chemotherapies. The physician must be aware of each of these tests, and ordering all of these tests would be prohibitively expensive and time consuming. Most precision medicine tests are currently performed in centralized labs, sometimes requiring samples to be sent vast distances for testing, especially from hospitals. If a physician requires multiple tests done by different companies, this is especially problematic since a single, often limited, sample or dataset may need to be submitted to a number of disparate geographic locations. As a result, biohazard risk is increased, the risk of losing sample or data is enhanced, and costs multiply. The time to acquire and integrate all of the test results may be protracted, adversely affecting clinical care.

### SUMMARY

Embodiments of systems and methods disclosed herein guide physicians and/or patients to select diagnostic tests that are most likely to be clinically impactful and cost effective, speeding up and streamlining the decision-making process. The systems and methods may provide a means to access evidence for each test's performance characteristics and clinical utility, including informing a user about stage of a regulatory clearance and insurance coverage. The embodiments of the present disclosure may identify tests that can be bundled together to reduce the need to transport samples to multiple central labs, and for cost efficiency. The embodiments of the present disclosure may include an analysis engine providing the capability to rapidly process complex data sets for analysis and interpretation, delivering a test report within a short period of time, and the embodiments of the present disclosure may enable the de-centralization of some testing, limiting the need to transport samples over vast distances, enhancing the efficiency of testing and reporting test results.

Some embodiments of the systems and methods disclosed herein guide test selection, provide centralized test ordering and interpretation, generate corresponding test results or combinations thereof. The embodiments of the present disclosure may include a clinical test recommendation tool, which may comprise a survey and a clinical test library, as well as be linked to a laboratory information system (LIS), a radiology information system (RIS), and/or an electronic medical record (EMR) system.

In some embodiments of the present disclosure may, a system for use by a user on a network comprises: a user terminal for obtaining one or more instructions from the user; two or more data pre-processing modules (DPPMs), each DPPM for obtaining a sample comprising raw medical test data and/or receiving raw medical test data and converting the medical test data to a data matrix; and an analysis engine comprising a plurality of diagnostic tests, the analysis engine for: performing a diagnostic test from the plurality of diagnostic tests on each of the data matrices and providing a diagnostic test result, providing one or more recommendations based on the diagnostic test result, and producing a report comprising the diagnostic test results and the one or more recommendations, wherein the user terminal, the DPPMs, and the analysis engine each comprise an interface to communicate over the network and the user can order one or more medical tests.

Some embodiments of the present disclosure relate to a system that operates on a cloud-based computer architecture, wherein the analysis engine comprises allocated scalable computing capacity.

Some embodiments of the present disclosure relate to a diagnostic test that comprises one or more machine learning (ML) methods.

Some embodiments of the present disclosure relate to a user terminal that comprises a user interface for operation of the system by a user.

In some embodiments of the present disclosure, the user terminal comprises a clinical test recommendation tool.

Some embodiments of the present disclosure relate to a clinical test recommendation tool that is for communicating with at least one of a LIS, a RIS and an EMR database or system.

In some embodiments, the clinical test recommendation tool comprises clinical data and a clinical test library of diagnostic tests.

In some embodiments of the present disclosure, the clinical test recommendation tool comprises a survey questionnaire.

In some embodiments of the present disclosure, the one or more recommendations comprises recommendations for additional tests.

In some embodiments of the present disclosure, the system further comprises a network storage device for storing the data matrix and results of the diagnostic tests.

In some embodiments of the present disclosure, the raw medical test data comprises one or more of a tissue sample, a fluid sample and imaging data.

In some embodiments of the present disclosure, the diagnostic test comprises immunohistochemical staining with one or more selected antibodies.

In some embodiments of the present disclosure, the diagnostic test comprises determining a molecular signature.

In some embodiments of the present disclosure, the diagnostic test comprises radiographic imaging analysis for characterizing features of a radiographic image.

In some embodiments of the present disclosure, the diagnostic test comprises mapping an epigenome for evaluating one or more epigenetic modifications to a genome.

In some embodiments of the present disclosure, the diagnostic test comprises evaluating chromatin accessibility.

In some embodiments of the present disclosure, the diagnostic test comprises evaluating a proteome.

In some embodiments of the present disclosure, the diagnostic test comprises evaluating a metabolome.

In some embodiments of the present disclosure, a method comprises the steps of: obtaining a sample comprising raw medical test data and/or receiving raw medical test data; converting the raw medical test data to a data matrix; performing a diagnostic classification based on the data matrix to provide a diagnostic test result for assisting in the evaluation of patient health; providing one or more recommendations based on the diagnostic test result; and producing a report based on the results of the diagnostic tests and the one or more recommendations.

In some embodiments of the present disclosure, the diagnostic test comprises a ML method.

In some embodiments of the present disclosure, the step of providing one or more recommendations comprises accessing at least one of a LIS, a RIS and an EMR database or system.

In some embodiments of the present disclosure, the step of providing one or more recommendations comprises accessing clinical data and a clinical test library.

In some embodiments of the present disclosure, providing one or more recommendations comprises administering a survey questionnaire.

In some embodiments of the present disclosure, providing one or more recommendations comprises recommendations for additional tests.

In some embodiments of the present disclosure, the method further comprises a step of storing the data matrix.

In some embodiments of the present disclosure, the method further comprises a step of storing results of the one or more diagnostic tests.

In some embodiments of the present disclosure, the raw medical test data comprises one or more of data from a tissue sample, data from a fluid sample, and imaging data.

In some embodiments of the present disclosure, the diagnostic test comprises immunohistochemical staining with one or more selected antibodies.

In some embodiments of the present disclosure, the diagnostic tests comprise determining a molecular signature.

In some embodiments of the present disclosure, the diagnostic test comprises radiographic imaging analysis for characterizing features on x-rays, computed tomography scans, magnetic resonance imaging cans and other radiographic studies.

In some embodiments of the present disclosure, the diagnostic test comprises mapping an epigenome for evaluating one or more epigenetic modifications to a genome.

In some embodiments of the present disclosure, the diagnostic test comprises evaluating chromatin accessibility.

In some embodiments of the present disclosure, the diagnostic test comprises evaluating a proteome.

In some embodiments of the present disclosure, the diagnostic tests comprises evaluating a metabolome.

### BRIEF DESCRIPTION OF THE DRAWINGS

For a more complete understanding of the disclosure, reference is made to the following description and accompanying drawings, in which:

FIG. 1 is a schematic diagram of a computerized guided diagnostic and integrated analysis system, according to some embodiments of the present disclosure;

FIG. 2 is a schematic diagram showing a simplified hardware structure of a computing device of the guided diagnostic and integrated analysis system shown in FIG. 1;

FIG. 3 is a schematic diagram showing a simplified software architecture of a computing device of the guided diagnostic and integrated analysis system shown in FIG. 1;

FIG. 4 is a schematic diagram showing a functional structure of the guided diagnostic and integrated analysis system shown in FIG. 1 implemented as a cloud-based system, according to an embodiment of the present disclosure;

FIG. 5A is a flowchart illustrating initial steps in an embodiment of a survey module;

FIG. 5B is a flowchart illustrating additional steps in the survey module of FIG. 5A relating to cancer-related diseases;

FIG. 5C is a flowchart illustrating additional steps in the survey module of FIG. 5A relating to respirology-related diseases;

FIG. 6 is a schematic diagram showing an example embodiment of a data pre-processing module;

FIG. 7 is a schematic diagram showing a cloud-based analysis engine; and

FIG. 8 is a flowchart illustrating the steps of an embodiment of a method of diagnostic and analysis.

### DETAILED DESCRIPTION

Unless otherwise defined, all technical and scientific terms used herein generally have the same meaning as commonly understood by one of ordinary skill in the art to which this disclosure pertains. Exemplary terms are defined below for ease in understanding the subject matter of the present disclosure.

The term “a” or “an” refers to one or more of that entity; for example, “a terminal” refers to one or more terminals or at least one terminal. As such, the terms “a” (or “an”), “one or more” and “at least one” are used interchangeably herein. In addition, reference to an element or feature by the indefinite article “a” or “an” does not exclude the possibility that more than one of the elements or features are present, unless the context clearly requires that there is one and only one of the elements. Furthermore, reference to a feature in the plurality (e.g., systems), unless clearly intended, does not mean that the systems or methods disclosed herein must comprise a plurality.

The expression “and/or” refers to and encompasses any and all possible combinations of one or more of the associated listed items (e.g. one or the other, or both), as well as the lack of combinations when interrupted in the alternative (or).

Some embodiments of systems and methods disclosed herein permit users to order tests, provide raw medical test data, and receive results therefrom. In some embodiments of systems and methods disclosed herein, diagnostic tests are selected, ordered and analyzed based on one or more multi-parametric assays to provide personalized clinical care, guiding the patient or the physician to select clinically relevant and appropriate test(s) that will inform clinical management. Provided guidance may comprise consideration of the following user inputs: patient characteristics, including demographics; clinical features related to the disease category; clinical questions that may inform clinical management; commercial availability of tests; sample types; and analytical platform. In some embodiments disclosed herein, a clinical test recommendation tool comprises a survey that can be administered by a patient or a physician and may comprise a clinical test library. The clinical test recommendation tool may also be linked to a laboratory information system (LIS) and/or a radiology information system (RIS) and/or an electronic medical record (EMR) system to further inform the guidance related to diagnostic testing. Recommendations may then be produced for tests that are personalized and appropriate for a particular patient.

In some embodiments disclosed herein, systems and methods comprise collecting and/or obtaining the medical test data. In some embodiments disclosed herein, this collecting or obtaining would comprise streaming medical test data from the instrument(s) conducting the sample analysis in real time as it is created, or as a bulk payload after the experiment completes a batch test or tests. In some embodiments disclosed herein, systems and methods provide guidance to users respecting what test(s) may be appropriate.

Embodiments of systems and methods disclosed herein provide an integrated system. In some embodiments disclosed herein, a user interface comprises a dashboard for a user to order one or more recommended tests suggested by the clinical test recommendation tool. In some embodiments disclosed herein, the system comprises an analysis engine, which may be cloud-based, connected to a machine learning (ML) function, a key vault library and a reporting module, as well as an auditing system and related databases. In some embodiments disclosed herein, the analysis engine comprises a bank of tests including test criteria and test purposes, which may be manually curated or manually/electronically curated. In some embodiments disclosed herein, the integrated analysis system further comprises one or more data pre-processing modules (DPPMs), which may accept test requests directly from the user interface or through the clinical test recommendation tool.

In some embodiments of systems and methods disclosed herein, a user is guided to identify one or more appropriate clinical test(s) then order, perform and process the test(s). In some embodiments disclosed herein, not all of these steps are required. For example, a user may just use the recommendation tool to identify appropriate tests. Alternatively, a user may just order, perform and process the test(s), without the recommendation tool.

A user may be a patient or lay person. For example, a patient may have a serious cancer with few treatment options who would like to identify and request a test that identifies actionable therapeutic targets.

A user may also be a clinician. For example, the clinician may have available some clinical information and laboratory information that could be used to predict a cerebrovascular accident (a stroke) or that raises concern for an underlying autoimmune disease. The raw medical data required for these queries may be provided to the system and the user selects tests based on specific questions. Alternatively, there may be a specific set of tests that are required. The raw medical data may be analyzed using corresponding predictive methods, and test reports may be produced.

A user may be in a lab where a biological sample is tested. For example, a tumor may be submitted to next generation sequencing (NGS) to identify genomic variants and transcriptomic features. Optionally, blood, plasma or serum samples may be analyzed by mass spectrometry to delineate proteomic or metabolomic features. The raw molecular data and linked clinical data may be pre-processed and uploaded to the cloud. A test may be selected, which corresponds to a specific predictive method. The data may then be analyzed in the context of that method and a test result is produced.

A user may be a radiologist who is having difficulty interpreting a radiological test. For example, on a CT scan, there is a lesion in the liver and it is difficult to determine whether it is benign or malignant. The Digital Imaging and Communications in Medicine (DICOM) data or a transformation thereof may be provided to the system. A predictive method designed to discriminate benign and malignant lesions of the liver may be used and a test result is produced.

In some embodiments disclosed herein, a user is guided to select from a clinical test library a clinically appropriate test, which may be based on criteria derived from a survey questionnaire. In some embodiments disclosed herein, the recommendation tool comprises a survey, which uses a branching method with questions, eliciting information that will guide selection of potentially appropriate tests from a clinical test library. Data from a LIS, a RIS and/or an EMR system can be used to inform the recommendation tool. Otherwise, the recommendation tool can be informed primarily from the survey questionnaire results. Data elicited from the survey may include one or more of the following:
Demographics: Age, Gender or Sex.
Sample(s) available: tissue biopsy, blood, urine, saliva, sputum, stool, imaging data, routine lab tests.
Disease categories: for example, cancer, hematology, dermatology, rheumatology, gastroenterology, respirology, nephrology, and cardiology.
Information types to be derived from reports and/or tests; for example, diagnostic, prognostic, or predictive information.

A clinical test library is a repository of commercially available tests and is regularly updated. In some embodiments disclosed herein, data related to each test will be used to match patient needs (based on recommendations of the recommendation tool survey) and the characteristics of each test. In other embodiments, a user and/or physician may select one or more tests without using the recommendation tool. The clinical test library comprises the following data elements: indications for the test (disease type, purpose of test), test category (early disease detection and early detection of recurrence; disease diagnosis and sub-classification; detection of treatment response; identification of actionable therapeutic targets; prognostication; and therapeutic prediction), type of sample (e.g. blood, tissue, urine, saliva, stool, imaging data, routine lab tests), levels of evidence, regulatory stage (e.g. research use only; laboratory developed test; regulatory clearance from specific jurisdictions), insurance coverage determinations, websites relates to tests and relevant published literature.

A guidance report is generated comprising a list of tests that may be suitable for the current clinical problem. The guidance report may include information that can be reviewed by the user and/or physician. The guidance report may include the following information: name of test, indications for test, scientific basis of test, sample type/data required for performing the test, analytical platform (e.g. whole transcriptome or targeted gene expression panel, mutation panel, single nucleotide polymorphism (SNP) panel, metabolomic assay, proteomic assay, computerized tomography (CT) scans, magnetic resonance imaging (MRI) scans, light microscopy imaging), clinical evidence and level of evidence, published literature, regulatory stage, insurance coverage determination(s), websites related to test, and instructions for ordering tests.

In some embodiments disclosed herein, a cloud-based analysis engine comprises a plurality of containers residing in the cloud (e.g. Docker® containers), each container comprising a diagnostic method corresponding to a specific diagnostic test. In some embodiments disclosed herein the methods comprise predictive models with specific clinical applications, such as disease classification, identifying events with common attributes (clustering), forecasting, identifying outliers, and tracking perturbations over time. Models can be based on any predictive or classification or regression method. Examples of predictive methods include linear regression, logistic regression, random forest, neural networks, gradient boosted models, K-means clustering, and generative adversarial networks. Methods corresponding to predictive models (and in turn comprising diagnostic tests) can be stored in encrypted and frozen formats in a cloud-based container, wherein “frozen” refers to code that is unalterable once put into a container, although updated versions can be installed and tracked. The cloud-based analysis engine may be a central repository for clinically useful methods, guiding users to select a diagnostic test or function, then upload relevant data, and run the selected method.

In some embodiments disclosed herein, the cloud-based analysis engine is linked to a clinical laboratory or radiology suite with a DPPM that communicates with the LIS or RIS or EMR, and concurrently processes data produced by analysis instrumentation. In some embodiments disclosed herein, systems and methods comprise analysis instrumentation and include obtaining the raw data. Examples of analysis instrumentation include a next generation sequencer, a mass spectrometer, a magnetic resonance imaging device, a computer tomography imaging device, and a microscopy imaging device. In some embodiment disclosed herein, the DPPM processes raw data from the analysis instrumentation, and converts the raw data to a data matrix in a format suitable for uploading to the cloud-based analysis engine. The system architecture disclosed herein enables scaling, with one or more DPPMs operatively connected with each user. The use of data matrices permits synchronous processing of batches comprising data from multiple patients.

Data used for predictive models may include molecular data such as mutation data, transcriptomic data, circular ribonucleic acid (RNA) expression, microRNA (miRNA) expression, long non-coding RNA (IncRNA) expression, epigenomic data, proteomic data, metabolomic data or any other molecular data that may be obtained from a subject's sample. Similarly, clinical, pathological and laboratory data may be included. Digital imaging data such as radiographic data or images from pathology slides can be used as input. The dashboard permits a user to request a diagnostic test, wherein the user is capable of selecting one or more diagnostic tests that processes the appropriate input data using relevant method(s).

Embodiments of systems and methods disclosed herein determine whether uploaded data, associated with one or more patients, matches known diagnostic signatures, and delivers reports to ordering physicians, labs, or other designated individuals. Embodiments of systems and methods disclosed herein are capable of rapidly interpreting complex and highly dimensional data to produce test reports which are transmitted to users and/or related parties. Some embodiments of systems and embodiments herein comprise sufficient security features that allow it to handle confidential and private data. In some embodiments disclosed herein, clinical reports with results and interpretation of the test(s) are generated, and test results are transmitted to ordering users, the EMR, the LIS, and/or the RIS.

Development of instrumentation with capability to concurrently quantify multiple parameters makes diagnostic tests increasingly complex. Multiple data points can be assembled to derive a test result with interpretation of such diagnostic tests requiring conversion of highly variable physical properties to a suitable digital format. Diagnostic tests can utilize very diverse types of data and have results in a wide variety of data formats.

For example, during the pathological examination of a tissue sample, it is common to perform immunohistochemical staining with selected antibodies to make a diagnosis. Staining intensity is an important consideration in the diagnosis. In recent years, this has been done using image analysis. The images are converted to DICOM, then staining intensity can be quantified on the tissue section as a whole or in selected regions. Similarly, immunofluorescent microscopic images can be assessed and quantified. Uses for this technology include establishing a tissue diagnosis, invasive tumor detection, and identification and characterization of biologically important features such as inflammatory infiltration.

Many molecular signatures are described that are based on the transcriptome. These include signatures that enable disease sub-classification, signatures related to specific biological functions, and prognostic signatures. A method of quantifying the level of expression of mRNA, miRNA and non-coding RNAs employs next generation sequencing (e.g. RNASeq, microarray, etc.). Sequence data are converted to a text file format, such as FASTQ, which must be further processed for downstream bioinformatics data analysis.

Radiographic imaging analysis is used to precisely characterize features from radiographic images such as X-rays, CT scans, MRI scans and other radiographic studies. DICOM images can be generated and images can be classified by patterns of features observed.

Some diagnostic tests are based on the capability to map the epigenome. These tests evaluate for epigenetic modifications to the genome, including histone modifications, chromatin remodeling, methylation and/or hydoroxymethylation of cytosine bases. Epigenetic modifications to the genome reflect environmental exposures and genetic influences of disease. Epigenome can be characterized by high-throughput technologies such as a methylation array, which interrogates methylation sites quantitatively. Methylation data may be expressed as beta values (ratio of the methylated probe intensity and the overall intensity, which is the sum of methylated and unmethylated probe intensities). Other technologies will have different data output formats.

Chromatin accessibility can be evaluated using a technology called assay for transposase-accessible chromatin with high-throughput sequencing (ATAC-Seq), which employs next generation sequencing. Identification of patterns related to accessible deoxyribonucleic acid (DNA) regions has been found to be useful for disease sub-classification and for interrogation of disease biology. Data produced requires a count matrix with number of reads per open chromatin region. Another technology may be chromatin immunoprecipitation sequencing (ChIP-sequencing), which combines chromatin immunoprecipitation with parallel DNA sequencing for analyzing protein interactions with DNA.

Proteomics is used for disease diagnosis, sub-classification and prognosis. There are many technologies available for the targeted or untargeted analysis of the proteome, including identification of post-translational modifications. Untargeted proteomics employs various types of mass spectrometry, which creates a “spectrum” of features such as mass-to-charge ratio; features correspond to different protein fragments. Peak size corresponds to protein abundance. Targeted proteomics includes antibody-based quantification of proteins.

Metabolomics has been used for disease diagnosis, sub-classification and prognosis. Various mass spectrometry-based platforms are employed to characterize the metabolome, depending on the physicochemical characteristics of the metabolites of interest. As in untargeted proteomics, spectra are generated with features that correspond to metabolites; peak size corresponds to quantity.

Any of these radiographic and molecular features can be integrated to create a more comprehensive understanding of disease sub-classification and related biology. To benefit from such integration, users require both understanding of and convenient access to the different tests. Patients generally have difficulty finding tests that will provide useful information for their health problems, as tests are performed by various companies in disparate locations and have limited access to information on the potential value of each test. Medical professionals generally have difficulty keeping up with the rapidly expanding list of available tests, often not fully understanding the science for each test, and ordering tests from multiple different vendors is time consuming.

Generally, ML methods are required to interpret these multi-parametric tests and are customized for the specific indication and/or purpose of the test. To implement such methods, the raw medical data produced by each test should be reduced to a simplified format that can be used as input for the methods. With the rapid development and expansion of these complex tests, it is increasingly difficult to coordinate testing, as tests are typically done in very specialized facilities. One solution is to centralize data interpretation. This is difficult because of the diversity of data formats that is produced by each test. In some embodiments disclosed herein, a DPPM, which reduces each data format to a simplified matrix that will act as input for the ML methods housed in a central hub.

### Examples

The following examples illustrate how users may benefit from the embodiments of the present disclosure.

Example 1: A patient has a (virtual) CT colonoscopy to screen for colorectal cancer. There is a lesion in the colon that could represent a polyp or a cancer or perhaps just a piece of stool. The radiologist's uncertainty prompts them to access an ML method residing in the engine. The data from the DICOM file is converted to an analyte matrix by the pre-processing module, then the data are uploaded to the analysis engine. The lesion has the characteristics of a cancer. A colonoscopy is performed, which confirms this. The patient undergoes resection, pathology confirms the diagnosis. RNA is extracted from the tumor, then submitted to whole transcriptome RNASeq to evaluate prognosis. Two separate tests are performed based on the whole transcriptome sequencing data, requiring processing through two separate methods. One test determines that the prognosis is poor, and a second test determines that this molecular subtype has a high incidence of lymph node metastases. The pathologist did not identify lymph node metastases by visual examination, so they stained the lymph nodes for cytokeratin and then created images stored in a DICOM file format. The data were pre-processed, then uploaded to the engine to access an ML method with the capability to identify extremely small metastases. Two micrometastases are identified. The patient is referred to oncology for consideration for chemotherapy. The oncologist accesses the guide to identify tests that would help to determine the best chemotherapy regimen. There are five chemosensitivity tests, each evaluating the appropriateness of different chemotherapy regimens. Two are based on RNASeq, two are based on evaluating the methylome, and one is based on ATAC-Seq. Whole transcriptome data from the first RNASeq are processed through two separate ML methods to perform the first two chemosensitivity tests. DNA are isolated from the tumor and then submitted to next generation sequencing to perform methylation sequencing and ATAC-Seq. The diverse data formats produced by the tests are reduced to simplified analyte matrices, then analyzed on the analysis engine with the appropriate diagnostic methods.

Example 2: A patient's health is declining. The related signs and symptoms are non-specific, and these include progressive and generalized weakness, muscle loss, a minor decline in cognitive function, and sore joints. The physician consults the guide, which suggests blood tests to analyze the proteome, the metabolome and the methylome. The diverse and complex data formats are simplified and reduced to an analyte matrix. Data from each of these tests are together uploaded to the analysis engine. Together, the data suggest that the symptoms are from Lyme disease. There is no evidence of an autoimmune etiology.

Example 3: A patient has a mammogram that demonstrates an indeterminate lesion in the right breast. The radiologist recommends an MRI scan. This is also indeterminate based on visual examination by the radiologist. The radiologist accesses the clinical test recommendation tool, which provides instructions to upload the DICOM data from the MRI to access an ML method that will aid in characterizing the lesion. The test result describes that the lesion has a high likelihood of being malignant. An excisional biopsy is performed, which demonstrates ductal carcinoma in situ. The pathologist orders special stains, including cytokeratin, estrogen receptor, progesterone receptor, HER2 receptor. The pathologist would like to ensure that there is no invasive component and uploads the DICOM image to access an ML image analysis. This demonstrates an invasive component. Treatments are then customized for invasive breast cancer. The clinical test recommendation tool provides a means to analyze the transcriptome for biomarkers that aid in disease sub-classification, as well as prognosis. Additional tests are recommended for consideration. Blood is analyzed for circulating tumor cells. The serum proteome and metabolome are analyzed to estimate the likelihood of occult metastatic disease. Chemosensitivity is evaluated using a test based on ATAC-Seq. The data for all of these tests are converted to data matrices by the pre-processing module. The data in the matrices are uploaded to the central hub to access corresponding classification methods residing within the system.

Example 4: A patient has a malignant lesion in the liver and routine pathology cannot definitively determine the type of cancer. The physician completes the survey, which determines that the 58-year-old male has a cancer of unknown origin, and that locoregional treatment (e.g. surgery, radiation, ablation) and systemic therapy are treatment considerations. The clinical test recommendation tool produces a report that recommends tests including whole transcriptome analysis using RNASeq. The tests recommended are tests designed to determine the cancer type, prognosis, and sensitivity to specific chemotherapy agents. RNASeq data comprised of sequenced reads stored in FASTQ files are processed by the relevant DPPM. A gene expression matrix is produced, then uploaded to the cloud-based analysis engine for further processing and producing predictions by applying selected methods to the data matrix (which in this case corresponds to a gene expression matrix). A test report is generated that describes the cancer type, the prognosis, and sensitivity to relevant chemotherapy agents. These tests are all based on gene expression signatures.

Example 5: A patient with breast cancer underwent surgery, and the physician would like to know the prognosis. The physician completes the survey comprising the recommendation tool. The recommendation tool allows the physician to input staging data. Several tests based on gene expression signature are recommended, and tests related to chemotherapy sensitivities are also recommended. They physician selects a test for prognosis that is based on a panel of genes quantified on a cDNA microarray. The microarray data and important clinical features are converted to a data matrix, specifically an analyte matrix, by the relevant DPPM. The combination of demographic data and staging data enhance the prognostic accuracy of the genomic risk.

Example 6: A radiologist identifies a pancreatic mass on CT scan. It is suspicious for pancreatic adenocarcinoma, but it may also represent a benign diagnosis such as autoimmune pancreatitis. They would like to access an AI (deep learning) method that provides an estimate of the likelihood that the tumor is malignant. The radiologist is guided through a series of questions in the recommendation tool. Tests corresponding to methods that process DICOM data and distinguish benign and malignant pancreatic tumors are recommended. DICOM data are processed through the DPPM, an imaging data matrix is produced, and data are uploaded to the cloud-based analysis engine. The features are compared to a diagnostic signature that was previously identified by artificial intelligence that is housed in an encrypted cloud-based container. The features of the DICOM image are compared to the signature of various benign and malignant masses of the pancreas, and it is determined that the mass likely represents a pancreatic adenocarcinoma.

Example 7: A cardiologist has a patient who is considered at high risk for myocardial infarction. The clinical test recommendation tool recommends a test that involves monitoring a set of clinical and laboratory variables serially over time. The test is based on a subtle combination of perturbations that predict a myocardial infarction. Once the test is ordered, data are acquired from the Electronic Medical Records (EMR) or from a wearable activity/fitness tracker (e.g. Apple® watch, Garmin® watch), and the DPPM transforms the data to a data matrix. The data are processed on the cloud-based analysis engine. A report is generated that depicts whether the combination of perturbations (the signature) raises concern over an impending myocardial infarction.

Example 8: A patient has been diagnosed with a rare cancer type, and there are very few data related to the efficacy of chemotherapy. The oncologist has suggested administering a chemotherapy regimen that has some activity based on case reports. The patient is hesitant because the chemotherapy regimen has some concerning toxicities. The patient accesses the recommendation tool and completes the survey. The recommendation tool output is a report that describes specific tests recommended, test indications and characteristics, the stage of regulatory clearance of each test, related literature and publications, as well as insurance coverage. The recommendation tool recommends whole genome analysis (looking for actionable mutations) and transcriptome-based molecular pathway analysis (to identify upregulated and targetable molecular features). The patient shares the report with his oncologist for further discussion.

### Computing Architecture

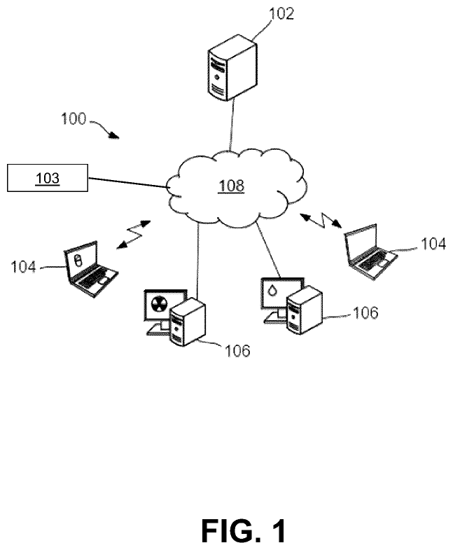

Embodiments of systems and methods of guided diagnostic testing and integrated analysis disclosed herein may be implemented on a variety of computer network architectures. Referring to FIG. 1, a computer network system for project and program management is shown and is generally identified using reference numeral 100. As shown, the computer network system 100 comprises one or more server computers or analysis engines 102 and a plurality of computing devices or terminals 104 for interfacing with a user, and a plurality of computing devices or terminals 106 for acquiring medical raw data and performing data pre-processing, each functionally interconnected by a network 108, such as the Internet, a local area network (LAN), a wide area network (WAN), a metropolitan area network (MAN), and/or the like, via suitable wired and wireless networking connections. In some embodiments disclosed herein, other devices 103 such as RIS, LIS, EMR, data warehouses, key vault libraries and audit devices may also be connected to the network 108.

The analysis engine 102 may be computing devices designed specifically for use as a server, and/or general-purpose computing devices acting as server computers while also being used by various users. Each analysis engine 102 may execute one or more server programs.

The terminals 104, 106 may be portable and/or non-portable computing devices such as laptop computers, tablets, smartphones, Personal Digital Assistants (PDAs), desktop computers, and/or the like. Each terminal 104, 106 may execute one or more client application programs which sometimes may be called “apps”.

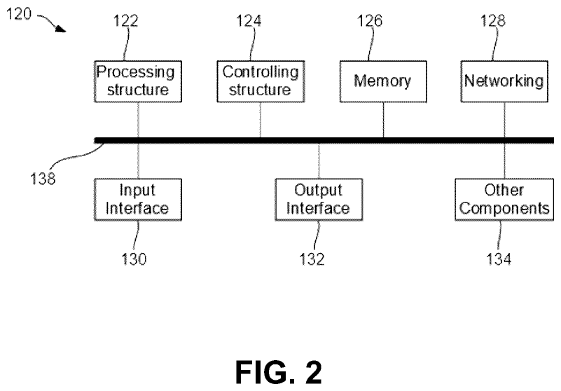

Generally, the analysis engines 102 and terminals 104, 106 have a similar hardware structure such as a hardware structure 120 shown in FIG. 2. As shown, the analysis engine 102 and terminal 104, 106 comprise a processing structure 122, a controlling structure 124, one or more non-transitory computer-readable memory or storage devices 126, a network interface 128, an input interface 130, and an output interface 132, functionally interconnected by a system bus 138. The analysis engine 102 and terminals 104, 106 may also comprise other components 134 coupled to the system bus 138.

The processing structure 122 may be one or more single-core or multiple-core computing processors (also called “central processing units” (CPUs)) such as INTEL® microprocessors (INTEL is a registered trademark of Intel Corp., Santa Clara, CA, USA), AMD® microprocessors (AMD is a registered trademark of Advanced Micro Devices Inc., Sunnyvale, CA, USA), ARM® microprocessors (ARM is a registered trademark of Arm Ltd., Cambridge, UK) manufactured by a variety of manufactures such as Qualcomm of San Diego, California, USA, under the ARM® architecture, or the like. When the processing structure 122 comprises a plurality of processors, the processors thereof may collaborate via a specialized circuit such as a specialized bus or via the system bus 138.

The processing structure 122 may also comprise one or more real-time processors, programmable logic controllers (PLCs), microcontroller units (MCUs), μ-controllers (UCs), specialized/customized processors and/or controllers using, for example, field-programmable gate array (FPGA) or application-specific integrated circuit (ASIC) technologies, and/or the like.

Generally, each processor of the processing structure 122 comprises necessary circuitries implemented using technologies such as electrical and/or optical hardware components for executing one or more processes as the implementation purpose and/or the use case may be, to perform various tasks. In many embodiments, the one or more processes may be implemented as firmware and/or software stored in the memory 126 and may be executed by the one or more processors of the processing structure 122. Those skilled in the art will appreciate that, in these embodiments, the one or more processors of the processing structure 122, are usually of no use without meaningful firmware and/or software.

For example, each processor of the processing structure 122 may comprise logic gates implemented by semiconductors to perform various computations, calculations, and/or processes. Examples of logic gates include AND gate, OR gate, XOR (exclusive OR) gate, and NOT gate, each of which takes one or more inputs and generates or otherwise produces an output therefrom based on the logic implemented therein. For example, a NOT gate receives an input (for example, a high voltage, a state with electrical current, a state with an emitted light, or the like), inverts the input (for example, forming a low voltage, a state with no electrical current, a state with no light, or the like), and output the inverted input as the output.

While the inputs and outputs of the logic gates are generally physical signals and the logics or processing thereof are tangible operations with physical results (for example, outputs of physical signals), the inputs and outputs thereof are generally described using numerals (for example, numerals “0” and “1”) and the operations thereof are generally described as “computing” (which is how the “computer” or “computing device” is named) or “calculation” or more generally, “processing”, for generating or producing the outputs from the inputs thereof.

Sophisticated combinations of logic gates in the form of a circuitry of logic gates, such as the one or more processors of the processing structure 122, may be formed using a plurality of AND, OR, XOR, and/or NOT gates. Such combinations of logic gates may be implemented using individual semiconductors, or more often be implemented as integrated circuits (ICs).

A circuitry of logic gates may be “hard-wired” circuitry which, once designed, may only perform the designed tasks. In this example, the tasks thereof are “hard-coded” in the circuitry.

With the advance of technologies, it is often that a circuitry of logic gates, such as the one or more processors of the processing structure 122, may be alternatively designed in a general manner so that it may perform various tasks according to a set of “programmed” instructions implemented as firmware and/or software and stored in the memory 126. In this example, the circuitry of logic gates, such as the one or more processors of the processing structure 122, is usually of no use without meaningful firmware and/or software.

Of course, those skilled in the art will appreciate that a processor may be implemented using other technologies such as analog technologies.

The controlling structure 124 comprises one or more controlling circuits, such as graphic controllers, input/output chipsets, and the like, for coordinating operations of various hardware components and modules of the analysis engine 102 and terminals 104, 106.

The memory 126 comprises one or more one or more non-transitory computer-readable storage devices or media accessible by the processing structure 122 and the controlling structure 124 for reading and/or storing instructions for the processing structure 122 to execute, and for reading and/or storing data, including input data and data generated by the processing structure 122 and the controlling structure 124. The memory 126 may be volatile and/or non-volatile, non-removable or removable memory such as RAM, ROM, EEPROM, solid-state memory, hard disks, CD, DVD, flash memory, or the like. In use, the memory 126 is generally divided into a plurality of portions for different use purposes. For example, a portion of the memory 126 (denoted as storage memory herein) may be used for long-term data storage, for example, for storing files or databases. Another portion of the memory 126 may be used as the system memory for storing data during processing (denoted as working memory herein).

The network interface 128 comprises one or more network modules for connecting to other computing devices or networks through the network 108 by using suitable wired or wireless communication technologies such as Ethernet, WI-FI® (WI-FI is a registered trademark of Wi-Fi Alliance, Austin, TX, USA), BLUETOOTH® (BLUETOOTH is a registered trademark of Bluetooth Sig Inc., Kirkland, WA, USA), Bluetooth Low Energy (BLE), Z-Wave, Long Range (LoRa), ZIGBEE® (ZIGBEE is a registered trademark of ZigBee Alliance Corp., San Ramon, CA, USA), wireless broadband communication technologies such as Global System for Mobile Communications (GSM), Code Division Multiple Access (CDMA), Universal Mobile Telecommunications System (UMTS), Worldwide Interoperability for Microwave Access (WiMAX), CDMA2000, Long Term Evolution (LTE), 3GPP, 5G New Radio (5G NR) and/or other 5G networks, and/or the like. In some embodiments, parallel ports, serial ports, USB connections, optical connections, or the like may also be used for connecting other computing devices or networks although they are usually considered as input/output interfaces for connecting input/output devices.

The input interface 130 comprises one or more input modules for one or more users to input data via, for example, touch-sensitive screens, touch-sensitive whiteboards, touch-pads, keyboards, computer nice, trackballs, microphones, scanners, cameras, and/or the like. The input interface 130 may be a physically integrated part of the analysis engine 102 or terminals 104, 106 (for example, the touch-pad of a laptop computer or the touch-sensitive screen of a tablet), or may be a device physically separated from but functionally coupled to, other components of the analysis engine 102 or terminal 104 (for example, a computer mouse). The input interface 130, in some implementation, may be integrated with a display output to form a touch-sensitive screen or a touch-sensitive whiteboard.

The output interface 132 comprises one or more output modules for output data to a user. Examples of the output modules include displays (such as monitors, LCD displays, LED displays, projectors, and the like), speakers, printers, virtual reality (VR) headsets, augmented reality (AR) goggles, and/or the like. The output interface 132 may be a physically integrated part of the analysis engine 102 or terminals 104, 106 (for example, the display of a laptop computer or a tablet), or may be a device physically separate from but functionally coupled to other components of the analysis engine 102 or terminal 104 (for example, the monitor of a desktop computer). The analysis engine 102 or terminal 104 may also comprise other components 134 such as one or more positioning modules, temperature sensors, barometers, inertial measurement units (IMUs), and/or the like.

The system bus 138 interconnects various components 122 to 134 enabling them to transmit and receive data and control signals to and from each other.

From the computer point of view, the analysis engine 102 and terminals 104, 106 may comprise a plurality of modules. Herein, a “module” is a term of explanation referring to a hardware structure such as a circuitry implemented using technologies such as electrical and/or optical technologies (and with more specific examples of semiconductors) for performing defined operations or processing. A “module” may alternatively refer to the combination of a hardware structure and a software structure, wherein the hardware structure may be implemented using technologies such as electrical and/or optical technologies (and with more specific examples of semiconductors) in a general manner for performing defined operations or processing according to the software structure in the form of a set of instructions stored in one or more non-transitory, computer-readable storage devices or media.

As a part of a device, an apparatus, a system, and/or the like, a module may be coupled to or integrated with other parts of the device, apparatus, or system such that the combination thereof forms the device, apparatus, or system. Alternatively, the module may be implemented as a standalone device or apparatus.

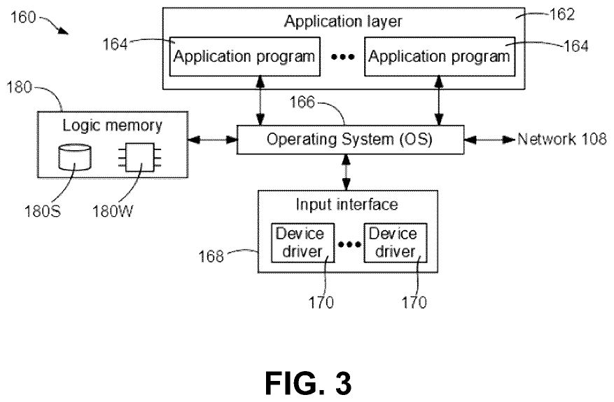

FIG. 3 shows a simplified software architecture 160 of the analysis engine 102 or terminals 104, 106. The software architecture 160 comprises an application layer 162, an operating system 166, a logical input/output (I/O) interface 168, and a logical memory 172. The application layer 162, operating system 166, and logical I/O interface 168 are generally implemented as computer-executable instructions or code in the form of software programs or firmware programs stored in the logical memory 172 which may be executed by the processing structure 122.

Herein, a software or firmware program is a set of computer-executable instructions or code stored in one or more non-transitory computer-readable storage devices or media such as the memory 126, and may be read and executed by the processing structure 122 and/or other suitable components of the analysis engine 102 and terminals 104, 106 for performing one or more processes. Those skilled in the art will appreciate that a program may be implemented as either software or firmware, depending on the design purposes and requirements. Therefore, for ease of description, the terms “software” and “firmware” may be interchangeably used hereinafter.

Herein, a process has a general meaning equivalent to that of a method, and does not necessarily correspond to the concept of computing process (which is the instance of a computer program being executed). More specifically, a process herein is a defined method implemented as software or firmware programs executable by hardware components for processing data (such as data received from users, other computing devices, other components of the analysis engine 102 or terminal 104, 106, and/or the like). A process may comprise or use one or more functions for processing data as designed. Herein, a function is a defined sub-process or sub-method for computing, calculating, or otherwise processing input data in a defined manner and generating or otherwise producing output data.

Alternatively, a process may be implemented as one or more hardware structures having necessary electrical and/or optical components, circuits, logic gates, integrated circuit (IC) chips, and/or the like.

Referring back to FIG. 3, the application layer 162 comprises one or more application programs 164 executed by or performed by the processing structure 122 for performing various tasks.

The operating system 166 manages various hardware components of the analysis engine 102 or terminal 104 via the logical I/O interface 168, manages the logical memory 172, and manages and supports the application programs 164. The operating system 166 is also in communication with other computing devices (not shown) via the network 108 to allow the application programs 164 to communicate with programs running on other computing devices. As those skilled in the art will appreciate, the operating system 166 may be any suitable operating system such as MICROSOFT® WINDOWS® (MICROSOFT and WINDOWS are registered trademarks of the Microsoft Corp., Redmond, WA, USA), APPLE® OS X, APPLE® iOS (APPLE is a registered trademark of Apple Inc., Cupertino, CA, USA), Linux, ANDROID® (ANDROID is a registered trademark of Google Inc., Mountain View, CA, USA), or the like. the analysis engine 102 and terminal 104 of the computer network system 100 may all have the same operating system, or may have different operating systems.

The logical I/O interface 168 comprises one or more device drivers 170 for communicating with respective input and output interfaces 130 and 132 for receiving data therefrom and sending data thereto. Received data may be sent to the application layer 162 for being processed by one or more application programs 164. Data generated by the application programs 164 may be sent to the logical I/O interface 168 for outputting to various output devices (via the output interface 132).

The logical memory 172 is a logical mapping of the physical memory 126 for facilitating the application programs 164 to access. In this embodiment, the logical memory 172 comprises a storage memory area that may be mapped to a non-volatile physical memory such as hard disks, solid-state disks, flash drives, and/or the like, generally for long-term data storage therein. The logical memory 172 also comprises a working memory area that is generally mapped to high-speed, and in some implementations, volatile physical memory such as RAM, generally for application programs 164 to temporarily store data during program execution. For example, an application program 164 may load data from the storage memory area into the working memory area, and may store data generated during its execution into the working memory area. The application program 164 may also store some data into the storage memory area as required or in response to a user's command.

As described above, the processing structure 122 is usually of no use without meaningful firmware and/or software. Similarly, while the computer network system 100 may have the potential to perform various tasks, it cannot perform any tasks and is of no use without meaningful firmware and/or software. As will be described in more detail later, the computer network system 100 described herein, as a combination of hardware and software, generally produces tangible results tied to the physical world, wherein the tangible results such as those described herein may lead to improvements to the computer and system themselves.

In some embodiments disclosed herein, systems and methods of guided diagnostic testing and integrated analysis are configured to operate on a cloud-based computer architecture, wherein the analysis engines 102 are virtual computing environments comprising allocated scalable computer capacity, which may be called instances. In some embodiments, each analysis engine 102 may be a variable configuration of CPU, memory, storage, and networking capacity. In some embodiments, each analysis engine 102 is assigned a unique internet protocol (IP) address within the network 108. The processing, memory, storage, and networking capacity may be provided by a single physical analysis engine 102 or a more complex computer architecture comprising a plurality of interconnected service, storage, and network components. The cloud-based computer architecture may be provided by a number of cloud computer service platforms such as Amazon Web Services® or AWS® (Amazon Web Services and AWS are registered trademarks of Amazon Web Services, Inc., a subsidiary of Amazon of Seattle, Washington, USA), Microsoft Azure™ (Azure is a trademark of Microsoft Corporation of Redmond, Washington, USA) and Google Cloud Platform or GCP.

In embodiments disclosed herein, systems and methods of guided diagnostic testing and integrated analysis enable raw data from an analytical instrument to be pre-processed, then converted or transformed to a data matrix. Examples of analytical instruments include a thermal cycler, a next generation sequencer, a mass spectrometer, a complementary DNA (cDNA) microarray, a protein array, a magnetic resonance imaging (MRI) device, and a computed tomography (CT) scanner. Data from analytical instruments are converted to formats that are compatible with disease classification using a predictive method contained in the analysis engine 102. Data are collated in the matrix with patient identifiers. Simultaneous testing of multiple patients is enabled, and data arrays may contain data from multiple patients, enabling batches of tests to be run.

In some embodiments disclosed herein, data matrices are sent to the cloud-based analysis engine. The cloud-based analysis engine 102 interrogates the data for important features. Some embodiments disclosed herein are configured to handle data from different analytical platforms, including mutation and genomic sequence variant data, transcriptional data (mRNA and noncoding RNA), epigenomic data, proteomic data and metabolomic data. In some embodiments disclosed herein, performance of tests may be enhanced by enrichment with clinical data and digital radiographic data can be analyzed.

In some embodiments disclosed herein, the cloud-based analysis engine 102 comprises a repository of clinically important predictive methods and may contain a plurality of containers or vaults in a ML environment. The containers may be organized in a container library with each container comprising a diagnostic method, are key encrypted and contain a locked predictive method. The containers may be version controlled to manage revisions or updates to the diagnostic method and/or locked predictive method. The predictive method(s) accessed for data analysis depend on the test(s) ordered. Each test may be based on complex and potentially highly dimensional data that may require deployment of specific predictive methods for test interpretation. In some embodiments disclosed herein, test reports are automatically generated and delivered.

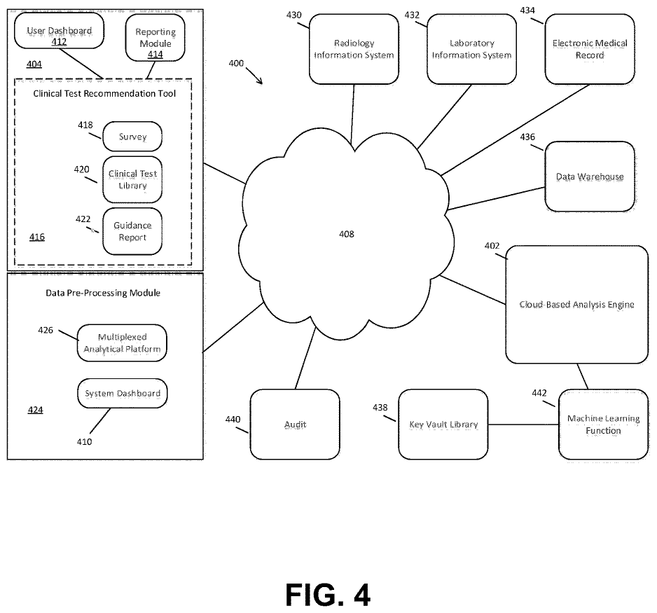

Referring to FIG. 4, in an embodiment disclosed herein, a system 400 comprises a cloud-based analysis engine 402 and one or more user terminals 404, one or more system terminals or DPPMs 424 connected over a network 408. In some embodiments disclosed herein, each user terminal 404 comprises a user dashboard 412, a reporting module 414, and a clinical test recommendation tool, In some embodiments disclosed herein, each DPPM 424 comprises a multiplexed analytical platform 426 for analyzing one or more analyte matrices and a system dashboard 410.

In some embodiments disclosed herein, the clinical test recommendation tool 416 is accessible to the user by a secure browser accessible portal and comprises a survey module 418, a clinical test library 120, and a guidance report module 422. In some embodiments disclosed herein, the recommendation tool 416 is linked to a LIS 432, a RIS 430, and/or an EMR 434. The survey module 418 uses a series of questions that help to define diagnostic needs of a patient. In some embodiments disclosed herein, survey questions are posed to a user, such as but not limited to in the format of a branching method wherein an answer to a question determines the next question. The clinical test library 120 comprises information derived from a survey and is used to match diagnostic needs with the features of tests contained within the library. The clinical test library 120 may be manually curated and updated.

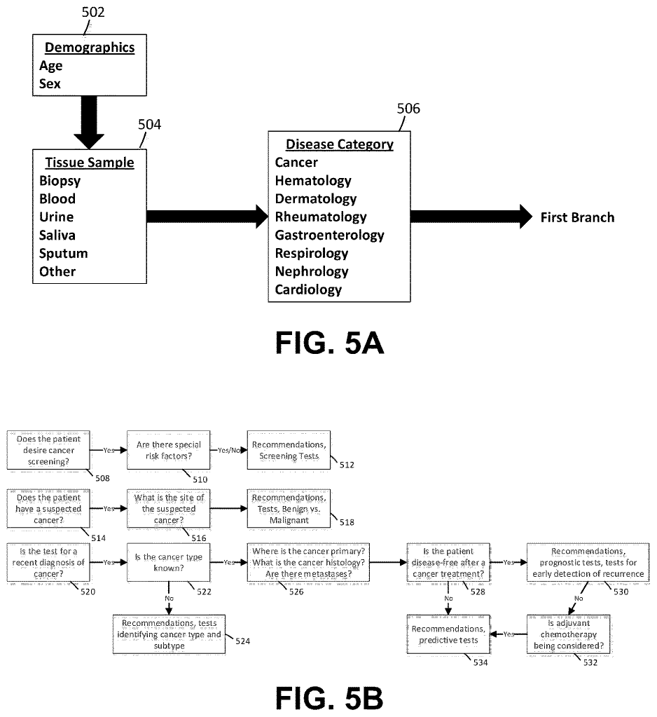

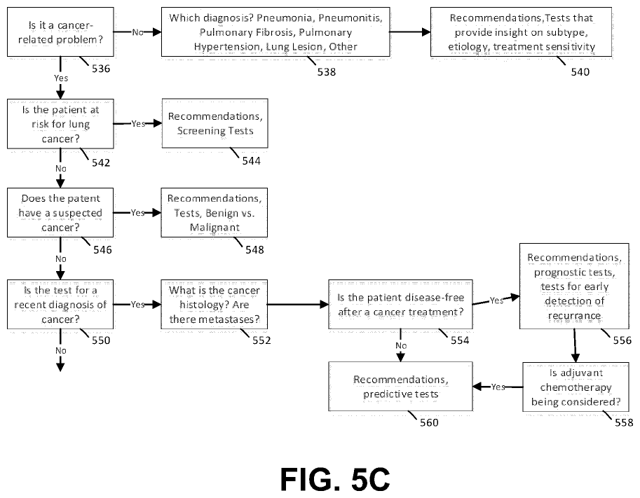

FIG. 5A to FIG. 5C illustrate survey questions of some exemplary embodiments disclosed herein. More specifically, FIG. 5A illustrates some initial questions relating to demographics, tissue sample and disease categories prior to entering more specific survey branches for specific disease groupings, such as FIG. 5B relating to cancer and FIG. 5C relating to respiratory diseases. Referring to FIG. 5A, at block 502, demographic data about a patient, such as age and sex, may be collected. At block 504, information relating to the type of tissue sample being provided, such as biopsy, blood, urine, saliva, or sputum, may be collected. At block 506, information relating to the disease category, such as cancer, hematology, dermatology, rheumatology, gastroenterology, respirology, nephrology, or cardiology, may be collected. Once initial information such as from blocks 502 to 506 is obtained, more specific survey branches may be entered.

FIG. 5B illustrates an exemplary embodiment of a survey branch and results reporting relating to cancer. At block 508, the survey module 418 prompts a user to answer whether the patient desires or requires cancer screening. If the patient does desire or require cancer screening, special risk factors are received at block 510 and recommendations and/or screening test results are provided at block 512. At block 514, the survey module 418 prompts a user as to whether the patient has a suspected cancer. If the patient does have a suspected cancer, the site of the suspected cancer is received at block 516 and recommendations, test results, and/or results as to whether the growth is benign or malignant is provided at block 518. At block 520, the survey module 418 prompts the user whether the test relates to a recent diagnosis of cancer. If the test does relate to a recent diagnosis of cancer, the survey module 418 prompts the user to answer whether the cancer type is known at block 522. If it is not known, recommendations as well as test results identifying cancer type and subtype are provided at block 524. If it is known, information relating to whether the cancer is primary, whether there is cancer histology, and/or whether there are metastases are received at block 526. The survey module 418 may then prompt the user to answer whether the patient is disease-free after a cancer treatment at block 528. If the patient is not disease-free after treatment, recommendations and predictive tests are provided at block 534. If the patient is disease-free after treatment, recommendations, prognostic test results, and/or test results for early detection of recurrence are provided at block 530. At block 532, the survey module 418 prompts the user as to whether adjuvant chemotherapy is being considered. If adjuvant chemotherapy is being considered, recommendations and predictive test results are provided at block 532.

FIG. 5C illustrates an exemplary embodiment of a survey branch relating to respirology. At block 536, the survey module 418 prompts a user to answer whether the problem is cancer-related. If the problem is not cancer-related, the diagnosis, such as pneumonia, pneumonitis, pulmonary fibrosis, and/or pulmonary hypertension is obtained at block 538 and recommendations and test results to provide insight on subtype, etiology, and/or treatment sensitivity are provided at block 540. If the response to block 536 was affirmative, the survey module 418 prompts the user whether the patient is at risk for lung cancer at block 542 and, if the patient is at risk of lung cancer, recommendations and screening test results are provided at block 544. If the response to block 542 was negative, the survey module 418 prompts the user whether the patient has a suspected cancer at block 546 and if the patient has a suspected cancer, recommendations, test results, and/or results as to whether a growth is benign or malignant is provided at block 548. If the response to block 546 was negative, the survey module 418 prompts the user whether the test relates to a recent diagnosis of cancer at block 550. If yes, the survey module prompts the user if there is a cancer histology and/or metastases at block 552. At block 554, the survey module prompts the user if the patient is disease-free after a cancer treatment at block 554. If the patient is disease-free after treatment, recommendations, results of prognostic tests, and/or tests for early detection of recurrence are provided at block 556 and the user is prompted whether adjuvant chemotherapy is being considered at block 558. If adjuvant chemotherapy is being considered or the response to block 554 is negative, recommendations and/or results of predictive tests are provided at block 560.

In some embodiments disclosed herein, the guidance report module 422 produces a report that describes diagnostic tests that would be potentially beneficial to a patient. Clinical tests that are determined to be relevant to diagnostic needs of a patient are recommended, prioritizing tests where samples/data are readily available. In some embodiments disclosed herein, the guidance report also indicates what tests are contained in the cloud-based analysis engine 402. A user can then access the user dashboard 412 to order test(s). Upon submitting test requests, a laboratory or vendor can process the order to obtain the appropriate sample or data set to perform the test.

In some embodiments disclosed herein, the user dashboard 412 is for ordering tests and comprises a portal gateway to order diagnostic tests contained in the cloud-based analysis engine 402, as well as tests that are available at other labs, wherein through the portal, a user (e.g., laboratory personnel, physicians, radiologists, etc.) has access to a menu of diagnostic tests in the cloud-based analysis engine 402. A user (patient or physician) may be directed to the portal after completion of a survey. Alternatively, a user may be directed to the user dashboard 412 without accessing the clinical test recommendation tool 416. In both instances, a user may be able to specify diagnostic test(s) to be performed on data contained in a data matrix. That is, the diagnostic test(s) selected by the user directs the matrix file to the corresponding predictive method in a key vault. In some embodiments disclosed herein, ordering tests initiates the following processes: collection of personal and demographic information; consent to access the related sample and relevant medical information; organization of the testing process.

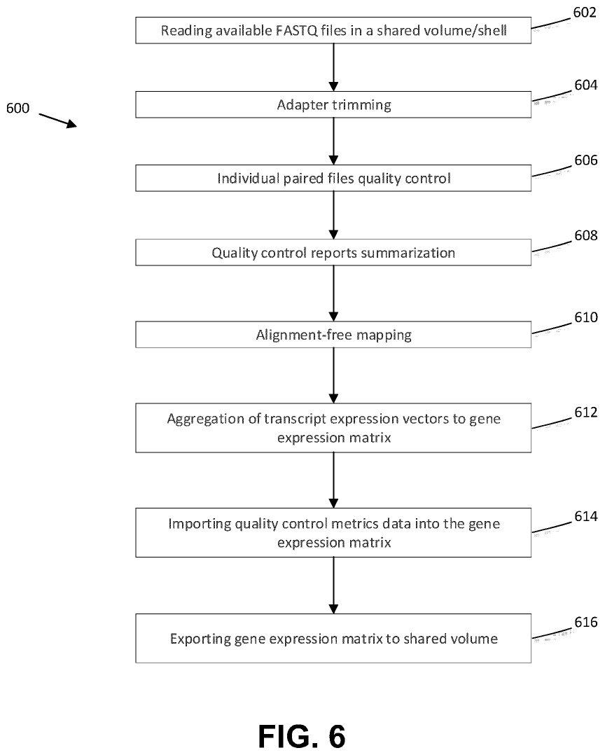

In some embodiments disclosed herein, the DPPM 424 is for processing raw medical data from analytical instrumentation (for example, a DNA/RNA sequencer, a mass spectrometer, and a diagnostic imaging instrumentation) to create a data matrix (or other structured file format) that will be uploaded to the cloud-based analysis engine 402. In some embodiments disclosed herein, the DPPM 424 connects to the user terminal 402 (e.g., a clinical lab or a radiology suite) and the cloud-based analysis engine 402 through a system dashboard 410. The DPPM 424 may be customizable, based on a particular use case. For example, the DPPM 424 is customizable based on the analytical platform and instrumentation and links to information systems that can be customized. The DPPM 424 may link with a LIS 432, a RIS 430, and/or an EMR 434 to acquire demographic, clinical and related data as well as patient identifiers. In some embodiments disclosed herein, the DPPM 424 creates a first data matrix file that contains important clinical information and patient identifiers. The DPPM 424 converts raw medical data from data from analytical instrumentation to a second data matrix file. For example, transcriptional FASTQ files from next generation sequencing are converted to a gene expression matrix; Whole Genome (WGS) or Whole Exome Sequencing (WES) FASTQ files are converted to VCF annotation files; mass spectrometry peaks are converted to a matrix file (or compressed version thereof) containing proteins or metabolites; or a DICOM file is converted to a matrix containing variables that inform the predictive method. The second data matrix file is separate from the first data matrix file(s) containing clinical information and patient identifiers. The first data matrix files and second data matrix files are sent to the cloud-based analysis engine 402 and processed using selected predictive methods. In some embodiments disclosed herein, data quality control (QC) analysis is performed prior to assembling data matrix files. Specifics for a QC check, including check types and criteria are determined based on the data type and specific usage. Referring to FIG. 6, an exemplary embodiment of a DPPM 424 for processing data from a sequencer. The method 600 comprises reading available FASTQ files from a shared volume or shell as input 602, performing adapter trimming 604, performing quality control on individual paired files 606, summarizing quality control reports 608, performing alignment-free mapping 610, aggregating transcript expression vectors to a gene expression matrix 612, importing quality control metrics data into the gene expression matrix 614 and exporting the gene expression matrix to a shared volume 616.

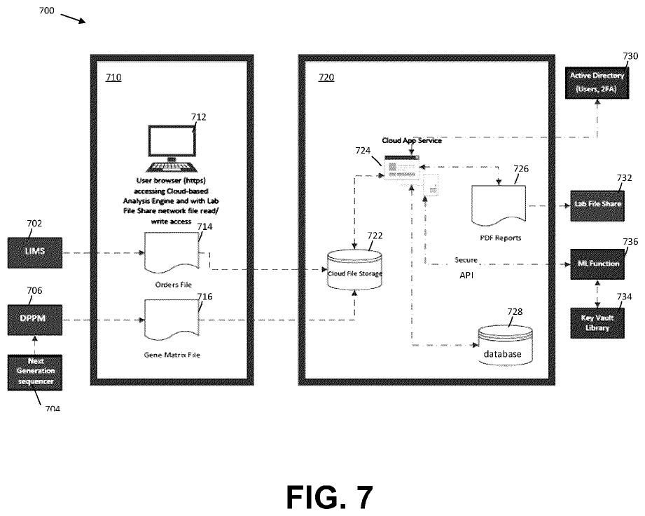

In some embodiments disclosed herein, a system dashboard 410 is for communicating with the user terminals 404 and the cloud-based analysis engine 402. In some embodiments disclosed herein, the system dashboard 410 comprises a portal gate to the user terminals 404 and the cloud-based analysis engine 402. In some embodiments disclosed herein, the portal is for uploading data matrix files created by the DPPM 424 to the cloud-based analysis engine 402. Referring to FIG. 7, an exemplary embodiment of an architecture of a cloud-based analysis engine 720 and its connection with a DPPM 706 is illustrated. More specifically, the relationship between the DPPM 706, cloud-based analysis engine 722, the ML function 736 and the key vault library 734 is illustrated. Referring to FIG. 7, the system 700 comprises a lab 710 and a cloud-based analysis engine 720. The lab 710 comprises a system dashboard 712, an orders file 714 (An orders file is information from an LIS, an RIS, or an EMR that links data in a data matrix to a specific individual or patient) and a gene matrix file 716 and is connected to the cloud-based analysis engine 720 as well laboratory information management system (LIMS) 702, a DPPM 706, wherein the DPPM 706 is interfaced with a next generation sequencer 704. The cloud-based analysis engine 720 comprises a cloud file storage 722, a cloud app service 724, a reporting module 726 and a database 728. The cloud-based analysis engine 720 further connected to an active directory 730, a lab file share 732 and a ML function 736.

In some embodiments disclosed herein, the cloud-based analysis engine 402 manages and controls the flow of data to an ML function that corresponds to a test ordered by the user. The cloud-based analysis engine 402 may be accessible through the browser-accessible portal, controls and audits data flow to ensure compliance with applicable standards including ISO13485, Health Insurance Portability and Accountability Act (HIPAA), Personal Information Protection and Electronic Documents Act (PIPEDA), General Data Protection Regulation (GDPR) and regulatory standards specific to the user's jurisdiction (e.g. Food and Drug Administration (FDA) in the U.S., and Health Canada in Canada). The cloud-based engine 402 may be connected to a ML function 442 comprising one or more containers. Once a diagnostic test is selected, a container of the ML function 442 identifies which method is relevant, then requests access to the method-specific secret key required to decrypt the method corresponding to the test. Each diagnostic method is “frozen” and may include additional QC checks. In some embodiments disclosed herein, the cloud-based analysis engine 402 comprises version control in cases where diagnostic methods are updated. In some embodiments disclosed herein, the cloud-based analysis engine 402 provides information to the reporting module 414, instructing the reporting module 414 respecting what test results should be generated. The ML function 442 is a set of one or more containers (or pods), each one capable of executing a prediction or classification task based on the requested test, by running the relevant method for the requested test. In some embodiments disclosed herein, the ML function 442 has access to a key vault library 438.

In some embodiments disclosed herein, the key vault library 438 is a library of cloud-based key vaults managed and administered by the cloud-based analysis engine 402. The key vault library 438 is called by the ML function 442. Access to each key vault is enabled using a secret key shared only with the ML function 442 which enables the encrypted diagnostic method to be decrypted for the purpose of implementing the selected diagnostic test.

In some embodiments a network storage device or a data warehouse 436 is connected to the network 408, containing data related to system access and performance, as well as data contained in data matrices.

In some embodiments disclosed herein, a reporting module 414 produces patient-specific test reports for providing an interpretation of the results of running the data through the diagnostic method. The reporting module 414 delivers patient test reports to the user and to specified recipients.

In some embodiments disclosed herein, an auditing system 440 logs and records the flow of data, including test orders, data processed through the DPPM 424, analysis of DPPM-processed data, ML function 442 logs, cloud-based analysis engine 402 logs, and generation/delivery of test reports.

Embodiments disclosed herein are scalable, having the capacity to process large numbers of tests from various locations, and also has the capability to accommodate a library of hundreds of thousands of clinically important predictive methods.

Embodiments disclosed herein comprise important security features essential for clinical diagnostics. In some embodiments disclosed herein, systems and methods are ISO13485 compliant, which is necessary for regulatory approval, as well as compliant with applicable privacy legislation (ensuring data privacy and security for safeguarding medical information). In some embodiments, locked diagnostic methods reside on a secure server, protected by their own encryption keys.

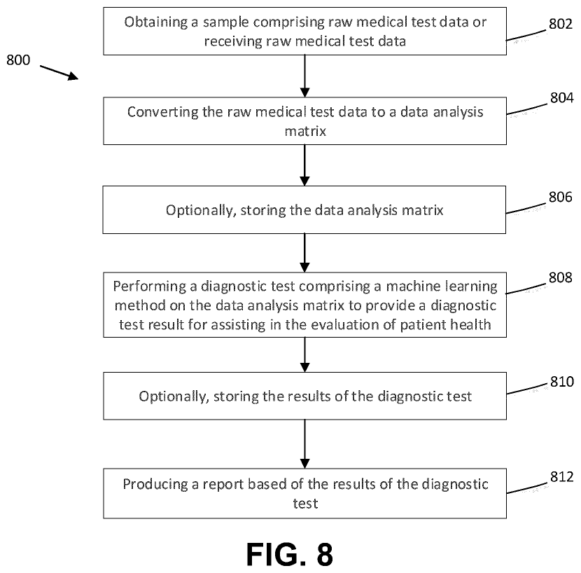

FIG. 8 is a flowchart showing the steps of a method 800, according to one embodiment of the present disclosure. The method 800 begins with obtaining a sample comprising medical test data or receiving raw medical test data (step 802). At step 804, raw medical data is converted to a data matrix. Optionally at step 806, the data matrix is stored. At step 808, a diagnostic test is performed using a ML method on the data matrix to provide a diagnostic test result for assisting in the evaluation of patient health. Optionally at step 810, the results of the diagnostic tests is stored. At step 812, a report based on the results of the diagnostic test is produced.

Although a few embodiments have been shown and described with reference to the accompanying drawings, it will be appreciated by those skilled in the art that various changes and modifications can be made to those skilled in the art that various changes and modifications can be made to these embodiments without changing or departing from their scope, intent, or functionality as defined by the appended claims. The terms and expressions used in the preceding specification have been used herein as terms of description and not of limitation, and there is no intention in the use of such terms and expressions of excluding equivalents of the features shown and described or portions thereof.

What is claimed is:

1. A system for use by a user on a network, the system comprising:
a user terminal for obtaining one or more instructions from the user;
two or more data pre-processing modules (DPPMs), each DPPM for receiving raw medical test data and converting the medical test data to a data matrix; and
an analysis engine comprising a plurality of diagnostic tests, the analysis engine for:
performing a diagnostic test from the plurality of diagnostic tests on each of the data matrices and providing a diagnostic test result,
providing one or more recommendations based on the diagnostic test result, and
producing a report comprising the diagnostic test results and the one or more recommendations,
wherein the user terminal, the DPPMs, and the analysis engine each comprise an interface to communicate over the network and the user can order one or more medical tests.

2. The system of claim 1 operating on a cloud-based computer architecture, wherein the analysis engine comprises allocated scalable computing capacity.

3. The system of claim 1 or 2, wherein the diagnostic tests comprises one or more machine learning (ML) methods.

4. The system of any one of claims 1 to 3, wherein the user terminal comprises a user interface for operation of the system by a user.

5. The system of any one of claims 1 to 4, wherein the user terminal comprises a clinical test recommendation tool.

6. The system of claim 5, wherein the clinical test recommendation tool is for communicating with at least one of a laboratory information system, a radiology information system and an electronic medical record database.

7. The system of claim 5 or 6, wherein the clinical test recommendation tool comprises clinical data and a clinical test library of diagnostic tests.

8. The system of any one of claims 5 to 7, wherein the clinical test recommendation tool comprises a survey questionnaire.

9. The system of any one of claims 5 to 8, wherein the one or more recommendations comprises recommendations for additional tests.

10. The system of any one of claims 1 to 9, further comprising a network storage device for storing the data matrix and results of the one or more diagnostic tests.

11. The system of any one of claims 1 to 10, wherein the raw medical test data comprises one or more of a tissue sample, a fluid sample and imaging data.

12. The system of any one of claims 1 to 11, wherein the diagnostic test comprises immunohistochemical staining with one or more selected antibodies.

13. The system of any one of claims 1 to 12, wherein the diagnostic test comprises determining a molecular signature.

14. The system of any one of claims 1 to 13, wherein the diagnostic test comprises radiographic imaging analysis for characterizing features of a radiographic image.

15. The system of any one of claims 1 to 14, wherein the diagnostic test comprises mapping an epigenome for evaluating one or more epigenetic modifications to a genome.

16. The system of any one of claims 1 to 15, wherein the diagnostic test comprises evaluating chromatin accessibility.

17. The system of any one of claims 1 to 16, wherein the diagnostic test comprises evaluating a proteome.

18. The system of any one of claims 1 to 17, wherein the diagnostic test comprises evaluating a metabolome.

19. A method comprising the steps of:
receiving raw medical test data;
converting the raw medical test data to a data matrix;
performing a diagnostic test on the data matrix to provide a diagnostic test result for assisting in the evaluation of patient health;
providing one or more recommendations based on the diagnostic test result; and
producing a report based on the results of the diagnostic tests and the one or more recommendations.

20. The method of claim 19, wherein the diagnostic test comprises a ML method.

21. The method of claim 19 or 20, wherein the step of providing one or more recommendations comprises accessing at least one of a laboratory information system, a radiology information system and an electronic medical record database.

22. The method of any one of claims 19 to 21, wherein the step of providing one or more recommendations comprises accessing clinical data and a clinical test library.

23. The method of any one of claims 19 to 22, wherein providing one or more recommendations comprises administering a survey questionnaire.

24. The method of any one of claims 19 to 23, wherein providing one or more recommendations comprises recommendations for additional tests.

25. The method of any one of claims 19 to 24, further comprising a step of storing the data matrix.

26. The method of any one of claims 19 to 25, further comprising a step of storing results of the one or more diagnostic tests.

27. The method of any one of claims 19 to 26, wherein the raw medical test data comprises one or more of data from a tissue sample, data from a fluid sample, and imaging data.

28. The method of any one of claims 19 to 27, wherein the diagnostic tests comprise immunohistochemical staining with one or more selected antibodies.

29. The method of any one of claims 19 to 29, wherein the diagnostic tests comprise determining a molecular signature.

30. The method of any one of claims 19 to 30, wherein the diagnostic tests comprise radiographic imaging analysis for characterizing features on x-rays, computed tomography scans, magnetic resonance imaging cans and other radiographic studies.

31. The method of any one of claims 19 to 31, wherein the diagnostic tests comprise mapping an epigenome for evaluating one or more epigenetic modifications to a genome.

32. The method of any one of claims 19 to 32, wherein the diagnostic tests comprise evaluating chromatin accessibility.

33. The method of any one of claims 19 to 33, wherein the diagnostic tests comprise evaluating a proteome.

34. The method of any one of claims 19 to 34, wherein the diagnostic tests comprise evaluating a metabolome.
